(19) United States
(12) Patent Application Publication
(10) Pub. No.: US 2022/0211683 A1
(43) Pub. Date: Jul. 7, 2022

(54) NOVEL MATERIAL FOR SKIN WOUND CLOSURE AND SCAR PREVENTION

(71) Applicant: The University of Chicago, Chicago, IL (US)

(72) Inventors: Xiaoyang WU, Chicago, IL (US); Jian ZHANG, Chicago, IL (US)

(73) Assignee: The University of Chicago, Chicago, IL (US)

(21) Appl. No.: 17/595,098

(22) PCT Filed: May 8, 2020
(86) PCT No.: PCT/US2020/070036

§ 371 Date: Nov. 9, 2021

Related U.S. Application Data
(60) Provisional application No. 62/845,648, filed on May 9, 2019.

(51) Int. Cl. A61K 31/4439; A61K 9/00; A61K 41/00; A61K 47/36; A61K 47/34; A61K 47/32; A61K 47/10; A61P 17/02; A61K 9/50; A61L 27/54; A61L 27/60; A61L 27/26

(52) U.S. Cl. CPC A61K 31/4439; A61K 9/0014; A61K 41/00; A61K 47/36; A61K 47/34; A61K 47/32; A61L 2300/414; A61P 17/02; A61K 9/5031; A61L 27/54; A61L 27/60; A61L 27/26; A61K 47/10

(57) ABSTRACT

Methods and compositions for preventing or inhibiting the formation of scars are disclosed. The methods and compositions include a TGF-β inhibitor provided in a delayed release composition.

### CROSS-REFERENCE TO RELATED APPLICATIONS

This application claims the benefit of priority of U.S. Provisional Patent Application No. 62/845,648 filed May 9, 2019, which is hereby incorporated by reference in its entirety.

### STATEMENT OF GOVERNMENT SUPPORT

This invention was made with Government support under grant no.: R01OD023700, awarded by the National Institutes of Health. The Government has certain rights in the invention.

### BACKGROUND OF THE INVENTION

### 1. Field of the Invention

The present invention relates to the fields of medicine, organic chemistry, and pathology.

### 2. Description of Related Art

Scarring often occurs following deep trauma, severe burn injury, or surgical incision. It can have a profound impact on the quality-of-life of patients, and pose a great challenge to physicians. One method to overcome scarring is to inhibit collagen synthesis by blocking transforming growth factor-β (TGF-β). However, the timing of TGF-β inhibition is crucial, as blocking the signal prematurely will result in poor wound healing, while blocking after peak collagen synthesis will have limited scar-reduction effect. Hence, we developed a novel composite wound photogelation dressing for timed discharge of TGF-β inhibitor in vivo. Specifically, we designed a timed delay-release drug delivery capsule system with poly(lactic-co-glycolic acid) o-nitrobenzene derivative (PLGA-NB) through water-in-oil-in-water (W/O/W) emulsion evaporation method. An efficient phototriggered-imine-crosslinking reaction was then used to integrate the capsule into the wound tissue via hyaluronic acid o-nitrobenzene derivative (HA-NB/HA-CDH). This facile treatment, termed the in situ-formed timed-pulsatile-release dressing (ISTD), can effectively decrease fibroblast activity and collagen deposition at a precise time point, resulting in significance reduction in scar formation in mouse, rabbit, and porcine models.

Excluding very minor lesions, almost all wounds will lead to scarring of different degrees. Generally, skin wounds that heal within about two weeks with minimal collagen deposition will not form scars. But if the repair process takes longer than three to four weeks, various scars such as stretch marks, keloids, hypertrophic scars or atrophic scars can occur depending on the situations. Scars in the skin usually have inferior function compared to normal skin. For example, they don't have sweat glands and hair follicles, and they may cause symptoms of pruritus and pain.¹ In addition, scars can sometimes restrict range of motion, and present as a permanently visible reminder of the traumatic event.²

Past research on scar models has led to a profound understanding in the pathophysiology of scar formation. One of the main regulators of scarring is TGF-β, which is an attractive target for scar-reduction strategies since there are readily available inhibitors against TGF-β. However, early attempts at targeting TGF-β are unsuccessful as they disregard the critical timing issue of drug delivery. Indeed, Mustoe et al. have demonstrated that starting anti-TGF-β antibody treatments in the first week after wounding actually delays wound healing without reducing scarring, as TGF-β is required for early wound healing. In contrast, scar formation is reduced when TGF-β inhibition is initiated one week after wounding. Thus, there is a clinical need to create a dressing material that allows for pulsatile release of TGF-β inhibitors one week after application. Many other methods such as coating, multiple-compaction have been invented.⁴⁻⁸ While these methods may potentially be utilized for delivering TGF-β inhibitors at an optimal time point to reduce scarring, they have not been validated for this purpose. Moreover, they require sophisticated three-dimensional microtechnology equipment that limits its potential for clinical application.

### SUMMARY OF THE INVENTION

Herein, a novel drug delivery system is presented. The system comprises an in situ-formed timed-pulsatile-release dressing (ISTD), which can be integrated into the wound via in situ photo triggered-imine-crosslinking pre-gelling polymer to deliver at least one TGF-β inhibitor at an optimal time point to increase wound healing and reduce scarring. An emulsion-based preparation method is used to load the at least one TGF-β inhibitor inside polymer capsules.⁹⁻¹¹ External light irradiation, which is safe and has been widely used in clinical regenerative medicine, is then given to allow the capsules and pre-gelling polymers to quickly crosslink with the surrounding tissue surfaces through o-nitrobenzene and —NH₂ reaction.¹² In some aspects, the external light irradiation is provided at a wavelength of 365 nm. This mingled cross-linking method stabilizes the timed release capsules to confer even microdopant packing, leading to trailed cargo discharge in the wound area, which may enhance the therapeutic effect.¹³ Finally, the timed-degradation of the polymer barrier results in the pulsatile release of the at least one TGF-β inhibitor to achieve optimal scar reduction potential. The ISTD shows excellent timed controllability, biocompatibility and efficient tissue integration. Most importantly, the system can significantly reduce hypertrophic scar formation without delaying wound healing in mouse, rabbit, and porcine skin wounding models.

In some aspects, a method for reducing dermal wound scar formation in a subject in need thereof is disclosed. The method comprises administering to the wound a composition comprising at least one TGF-β inhibitor and a crosslinkable polymeric composition, and inducing crosslinking of the crosslinkable polymeric composition to form a crosslinked polymeric composition. Upon crosslinking the crosslinkable polymeric composition, the crosslinking composition covalently attaches to dermal tissue. Examples of crosslinkable polymeric compositions may be found in U.S. Patent Publication No. 2017/0313827, the entirety of which is incorporated by reference herein. In some aspects, the at least one TGF-β inhibitor is provided in a delayed release capsule. In embodiments, a delayed release capsule is formulated to delay release of at least one TGF inhibitor for a period ranging from one day to sixty days. In some embodiments, the delayed release capsule further comprises at least one wound healing agent selected from the group consisting of vascular endothelial growth factor (VEGF), epidermal growth factor, and at least one cytokine. The at least one cytokine may be selected from IL-7, IL-10, and other cytokines known in the art. In some embodiments, the dermal tissue is covered with a bandaging material subsequent to administering the scar formation-reducing composition. The delayed release capsule may comprise an o-nitrobenzyl functionalized polymer. In some embodiments, the o-nitrobenzyl functionalized polymer molecular weight may range from 1,000 to 100,000 Da. The o-nitrobenzyl functionalized polymer molecular weight may be 1,000, 2,000, 3,000, 4,000, 5,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000 Da, or any value in between the foregoing. In some embodiments, the o-nitrobenzyl functionalized polymer is o-nitrobenzyl functionalized poly(lactic-co-glycolic acid) (PLGA). In some embodiments, the o-nitrobenzyl functionalized PLGA molecular weight may range from 1,000 to 100,000 Da. The o-nitrobenzyl functionalized PLGA molecular weight may be 1,000, 2,000, 3,000, 4,000, 5,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000 Da, or any value in between the foregoing. In some embodiments, the PLGA lactic/glycolic acid ratio may range from 1:1,000 to 1,000:1. The PLGA lactic/glycolic acid ratio may be 1:1,000, 2:1,000, 3:1,000, 4:1,000, 5:1,000, 1:100, 2:100, 3:100, 4:100, 5:100, 6:100, 7:100, 8:100, 9:100, 1:10, 2:10, 3:10, 4:10, 5:10, 6:10, 7:10, 8:10, 9:10, 1:1, 10:1, 20:1, 30:1, 40:1, 50:1, 60:1, 70:1, 80:1, 80:1, 100:1, 200:1, 300:1, 400:1, 500:1, 600:1, 700:1, 800:1, 900:1, 1,000:1, or any range derivable therein. In some embodiments, the o-nitrobenzyl functionalized polymer is poly(lactic acid). In other embodiments, the o-nitrobenzyl functionalized polymer is poly(glycolic acid). In some embodiments, the poly(lactic acid) molecular weight may range from 1,000 to 100,000 Da. The poly(lactic acid) molecular weight may be 1,000, 2,000, 3,000, 4,000, 5,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000 Da, or any value in between the foregoing. In some aspects, the poly(glycolic acid) molecular weight may range from 1,000 to 100,000 Da. The poly(glycolic acid) molecular weight may be 1,000, 2,000, 3,000, 4,000, 5,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000 Da, or any value in between the foregoing. The delayed release capsule may further comprise additional components, including but not limited to polyvinyl alcohol (PVA) and polyethylene glycol (PEG). In some aspects, the PVA molecular weight may range from 100 to 5,000 Da. The PVA molecular weight may be 100, 200, 300, 400, 500, 600, 700, 800, 900, 1,000, 2,000, 3,000, 4,000, 5,000 DA, or any value in between the foregoing. In some embodiments, the PEG molecular weight may range from 1,000 to 5,000 Da. The PEG molecular weight may be 100, 200, 300, 400, 500, 600, 700, 800, 900, 1,000, 2,000, 3,000, 4,000, 5,000 Da, or any value in between the foregoing. In some embodiments, the at least one TGF-β inhibitor is selected from the group consisting of SB431542, LDN-193189, galunisertib (LY2157299), LY2109761, SB525334, LY 3200882, SB505124, pirfenidone, GW788388, LY364947, RepSox, LDN-193189, K02288, SD-208, LDN-214117, SIS3, vactosertib (TEW-7197), DMH1, LDN-212854, ML347, kartogenin, hesperetin, alantolactone, GC-1008, and LY550410. Lahn M. et al. and Yingling, J. M. et al. disclose TGF-β inhibitors that may be employed in the methods and compositions disclosed herein.^{39,40} The TGF-β inhibitors disclosed by Lahn M. et al. and Yingling, J. M. et al. are incorporated herein by reference. It is specifically contemplated that 1, 2, 3, 4, 5, 6, 7, 8, 9, 10 or more of these TGF-β inhibitors may be excluded from an embodiment described herein.

In some aspects, a method for increasing wound healing in a subject in need thereof is disclosed. The method comprises administering to the wound a composition comprising a crosslinkable polymeric composition and at least one wound healing agent selected from the group consisting of VEGF, epidermal growth factor, fibroblast growth factor, connective tissue growth factor, secretory leukocyte protease inhibitor, insulin-like growth factor, insulin-like growth factor binding protein, platelet derived growth factor, an agonist or antagonist of vascular endothelial growth factor, transforming growth factor (33, thymosin, and/or at least one cytokine, and inducing crosslinking of the crosslinkable polymeric composition to form a crosslinked polymeric composition. The VEGF family comprises five members, VEGF-A, placenta growth factor (PGF), VEGF-B, VEGF-C, and VEGF-D. Any or all of the VEGF family members may be used in the wound healing methods disclosed herein. The at least one cytokine may be selected from IL-7, IL-10, and other cytokines known in the art. In some aspects, the at least one wound healing agent is provided in a delayed release capsule.

In some embodiments, the crosslinkable polymeric composition comprises an o-nitrobenzyl functionalized polysaccharide, polypeptide, protein, or hydrophilic or water-soluble polymer. In some embodiments, the o-nitrobenzyl functionalized polysaccharide, polypeptide, protein, or hydrophilic or water-soluble polymer molecular weight may range from 1 kDa to 500 kDa. The o-nitrobenzyl functionalized polysaccharide, polypeptide, protein, or hydrophilic or water-soluble polymer molecular weight may be 1, 2, 3, 4, 5, 6, 7, 8, 9, 10, 20, 30, 40, 50, 60, 70, 80, 90, 100, 200, 300, 400, 500 kDa, or any value in between the foregoing. In some aspects, the crosslinkable polymeric composition further comprises a terminal amine-functionalized polysaccharide, polypeptide, protein, or hydrophilic or water-soluble polymer. In some embodiments, the terminal amine-functionalized polysaccharide, polypeptide, protein, or hydrophilic or water-soluble polymer molecular weight may range from 1 kDa to 500 kDa. The terminal amine-functionalized polysaccharide, polypeptide, protein, or hydrophilic or water-soluble polymer molecular weight may be 1, 2, 3, 4, 5, 6, 7, 8, 9, 10, 20, 30, 40, 50, 60, 70, 80, 90, 100, 200, 300, 400, 500 kDa, or any value in between the foregoing. In some aspects, the o-nitrobenzyl functionalized polysaccharide is o-nitrobenzyl functionalized hyaluronic acid. In some embodiments, the hyaluronic acid molecular weight may range from 1 kDa to 500 kDa. The hyaluronic acid molecular weight may be 1, 2, 3, 4, 5, 6, 7, 8, 9, 10, 20, 30, 40, 50, 60, 70, 80, 90, 100, 200, 300, 400, 500 kDa, or any value in between the foregoing. In some aspects, the terminal amine-functionalized polysaccharide is carbohydrazide-functionalized hyaluronic acid.

Some embodiments are directed to a composition. In some embodiments, the composition may be used for reducing dermal wound scar formation in a subject. The composition comprises a delayed release capsule comprising at least one TGF-β inhibitor and a crosslinkable polymeric composition, in some embodiments.

In some aspects, the crosslinkable polymeric composition comprises one or more crosslinkable polymers. In some aspects, the crosslinkable polymeric composition comprises an o-nitrobenzyl functionalized polysaccharide, polypeptide, protein, or hydrophilic or water-soluble polymer. In some embodiments, the o-nitrobenzyl functionalized polysaccharide is o-nitrobenzyl functionalized hyaluronic acid. In some aspects, the crosslinkable polymeric composition further comprises a terminal amine-functionalized polysaccharide, polypeptide, protein, or hydrophilic or water-soluble polymer. In some embodiments, the terminal amine-functionalized polysaccharide is carbohydrazide-functionalized hyaluronic acid. In some aspects, the crosslinkable polymeric composition comprises o-nitrobenzyl functionalized hyaluronic acid and carbohydrazide-functionalized hyaluronic acid.

Crosslinking of the crosslinkable polymeric composition may be accomplished by a variety of crosslinking methods known to those of skill in the art. In some embodiments, crosslinking comprises irradiating the crosslinkable polymeric composition with light from a light source to activate crosslinking reactions. In some aspects, the light source has an illumination wavelength in the range of 250 to 500 nm. The light source illumination wavelength may be 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500 nm, or any value in between the foregoing. In some embodiments, the illumination wavelength of the crosslinking-inducing light source is 365 nm. In some aspects, the crosslinkable polymeric composition is provided in solution. The crosslinkable polymeric composition solution may be provided at a concentration ranging from 0.1 to 50% by weight of crosslinkable polymeric composition per unit weight of water. In some aspects, a ratio of o-nitrobenzyl functionalized hyaluronic acid to carbohydrazide-functionalized hyaluronic acid ranges from 0.01:1 to 100:1. The ratio of o-nitrobenzyl functionalized hyaluronic acid to carbohydrazide-functionalized hyaluronic acid may be 0.01:1, 0.02:1, 0.03:1, 0.04:1, 0.05:1, 0.06:1, 0.07:1, 0.08:1, 0.09:1, 0.1:1, 0.2:1, 0.3:1, 0.4:1, 0.5:1, 0.6:1, 0.7:1, 0.8:1, 0.9:1, 1:1, 2:1, 3:1, 4:1, 5:1, 6:1, 7:1, 8:1, 9:1, 10:1, 20:1, 30:1, 40:1, 50:1, 60:1, 70:1, 80:1, 80:1, 100:1, or any range derivable therein. In some embodiments, the delayed release capsule comprises one or more TGF-β inhibitors.

The delayed release capsule may comprise about from 0.1 to 1,000 mg of each TGF-β inhibitor per 100 g of polymer. The delayed release capsule may comprise about from 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, 1.00 mg, or any value in between the foregoing. The delayed release capsule may comprise an o-nitrobenzyl functionalized polymer. In some embodiments, the o-nitrobenzyl functionalized polymer is o-nitrobenzyl functionalized poly(lactic-co-glycolic acid) (PLGA). In some embodiments, the PLGA lactic/glycolic acid ratio may range from 1:1,000 to 1,000:1. The PLGA lactic/glycolic acid ratio may be 1:1,000, 2:1,000, 3:1,000, 4:1,000, 5:1,000, 1:100, 2:100, 3:100, 4:100, 5:100, 6:100, 7:100, 8:100, 9:100, 1:10, 2:10, 3:10, 4:10, 5:10, 6:10, 7:10, 8:10, 9:10, 1:1, 10:1, 20:1, 30:1, 40:1, 50:1, 60:1, 70:1, 80:1, 80:1, 100:1, 200:1, 300:1, 400:1, 500:1, 600:1, 700:1, 800:1, 900:1, 1,000:1, or any range derivable therein. In some embodiments, the o-nitrobenzyl functionalized polymer is poly(lactic acid). In other embodiments, the o-nitrobenzyl functionalized polymer is poly(glycolic acid). The delayed release capsule may further comprise additional components, including but not limited to polyvinyl alcohol (PVA) and polyethylene glycol (PEG). In some embodiments, the at least one TGF-β inhibitor is selected from the group consisting of SB431542, LDN-193189, galunisertib (LY2157299), LY2109761, SB525334, LY 3200882, SB505124, pirfenidone, GW788388, LY364947, RepSox, LDN-193189, K02288, SD-208, LDN-214117, SIS3, vactosertib (TEW-7197), DMH1, LDN-212854, ML347, kartogenin, hesperetin, alantolactone, GC-1008, and LY550410.

The delayed release capsule o-nitrobenzyl functionalized polymer (e.g., PLGA, PLA, or PGA), the lactic/glycolic acid ratio of the o-nitrobenzyl functionalized PLGA, crosslinkable polymeric composition polymer molecular weight, and the crosslinkable polymeric composition polymer end groups may each be independently selected to achieve a desired composition physicochemical characteristic. Each of these features may be independently selected to adjust or modify physicochemical characteristics selected from the group consisting of glass transition temperature, delayed release capsule degradation rate, delayed release capsule release rate, crosslinked polymeric composition degradation rate, and TGF-β inhibitor release rate.

Embodiments also include methods of making such compositions. In some embodiments, a TGF-β inhibitor is combined or mixed a crosslinkable polymeric compound.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” Is is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value. The phrase “wound healing” refers to replacement of destroyed tissue by living tissue. The phrase “increasing wound healing” may refer to increasing the rate at which destroyed tissue is replaced by living tissue, or the phrase may refer to increasing the degree to which destroyed tissue is replaced by living tissue.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Schematic diagram. Capsules are fabricated by W/O/W emulsion method. It releases inside payload (at least one TGF-β inhibitor) in distinct, timed delayed bursts without leakage. After photo-gelation with HA-NB/HA-CDH and skin wound tissue, it can be applied as wound dressing to prevent scar hypertrophy without delaying wound healing.

FIG. 2A-B FESEM photograph of the capsules and the crushed Cap-Hollow capsules.

FIG. 3A-C Pulsatile release kinetics of timed release capsule. (a) In vitro cumulative release of encapsulated BSA from Cap-BSA capsule at 37° C. (b) In vivo max radiant efficiency change profile of Cap-FRET injected mice. Note that this has a relative less sharp release curve than in vitro release. It is due to the onset of release can vary marginally in individual mice even though each capsule exhibits a pulsatile discharge. (c) Images of mice collected with IVIS.

FIG. 4 Photographs of wounds before and after applying materials.

FIG. 5 Photocrosslinking of HA-NB/HA-CDH with capsule and the surrounding wound tissue. O-nitrobenzene group in the HA-NB/HA-CDH and the shell of capsule will be converted to o-nitrosobenzaldehyde groups under ultraviolet light, which leads to the integration of macromolecule, capsule and the surrounding wound surface by the crosslinking of o-nitrosobenzaldehyde groups and the —NH₂ group.

FIG. 6A-D Evaluation of scar on CD-1 mouse dorsal skin. (a) Dermal fibrosis, as assessed by the extent of collagen deposition in wounded site and uninvolved skin of mice. Numbers of (b) F4/80, (c) α-SMA, (d) CD4+ antibody and CD8+ antibody positive cells in each high power field of wounded site. * p<0.05, ** p<0.001.

FIG. 7A-C Representative histology examples of wounded mouse dorsal skin among the Cap-Hollow dressing, Cap-I dressing and Saline treatment. Paraffin sections of scar tissues collected on day 5 (a), 10 (b) and 15 (c) after surgery.

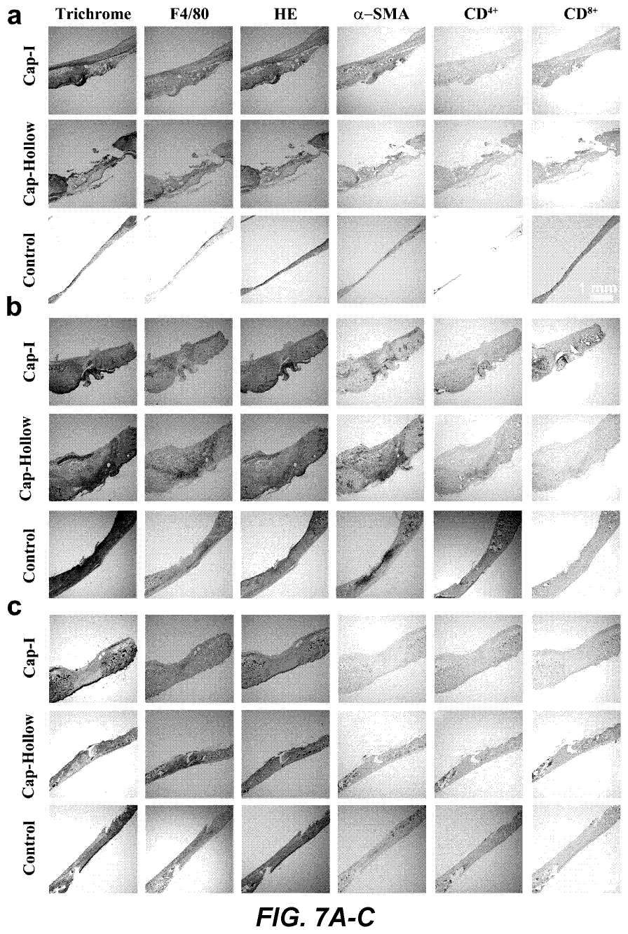

FIG. 8A-C Pictures of the rabbit ear and the measure method of scar elevation index (SEI). (a) Photographs of rabbit ear at different time points. (b) Histologic cross-section of the hypertrophic scar (Trichrome and H&E). (c) The SEI is the ratio of the whole dermal height (α+β), including the newly formed hypertrophied dermis height (α), to the underlying dermis height (β).

FIG. 9 Pictures of the treatment and the rabbit ear.

FIG. 10 Histologic cross-section of the hypertrophic scar formed 30 days after surgery (Trichrome and H&E).

FIG. 11 Histologic cross-section of the hypertrophic scar formed 60 days after surgery (Trichrome and H&E of the other 3 wounds which doesn't show in FIG. 7).

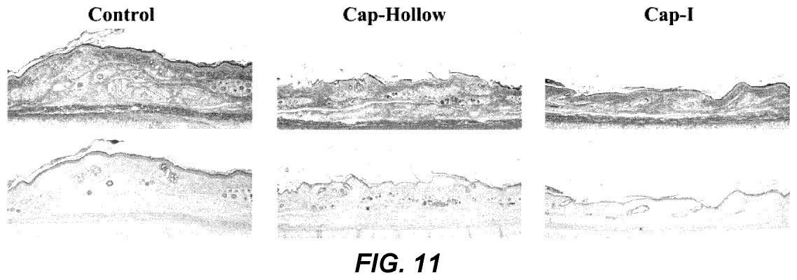

FIG. 12A-B Collagen deposition and SEI evaluation of scar on rabbit ear. * p<0.05, ** p<0.005.

FIG. 13 Light microscope photograph of the capsules.

FIG. 14 The cell viability against Cap-Hollow, Cap-I, Cap-BSA and Cap-FRET for 24 hours relative to untreated group, as determined by CCK-8 assay.

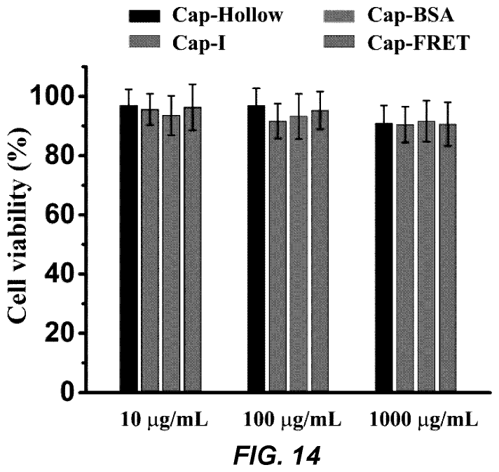

FIG. 15 Delivery of TGFβ inhibitor with PLGA-NB capsules can enhance skin wound closure while suppressing scarring. (a) Quantification of skin wound closure in porcine skin wounds after treatment with PLGA or PLGA capsules loaded with TGFβ inhibitor. (b) Haematoxylin and eosin (H/E) and trichrome staining of porcine skin sections treated with PLGA capsules or PLGA capsules loaded with TGFβ inhibitor.

FIG. 16A-C PLGA-NB delivery platform can promote scarless wound healing in porcine skin wound healing model. (a) Representative images of porcine skin wound healing and scar formation in differently treated groups. (b-c) Quantification of collagen deposition and SEI (scar elevation index) at different time points after wounding. All error bars represent SD.

### DETAILED DESCRIPTION OF THE INVENTION

The present disclosure provides embodiments that overcome the deficiencies of the prior art by providing compositions and methods for reducing scar hypertrophy. In some embodiments, methods and compositions involve inhibition of collagen synthesis by blocking transforming growth factor-β (TGF-β). One or more TGF-β inhibitors can be provided within a timed delay-release drug delivery capsule system that is integratable in wound tissue. The timed release capsule system can be integrated with a subject's wound tissue through a polymer crosslinking reaction that involves covalent binding of the crosslinked polymer to the wound tissue. The compositions and methods may include vascular endothelial growth factor, and/or epidermal growth factor to promote wound healing.

### Therapeutic Compositions

The methods disclosed herein may comprise administration of a combination of therapeutic agents, such as a first therapeutic agent for reducing dermal wound scar formation and a second therapeutic agent for reducing dermal wound scar formation. The methods disclosed herein may comprise administration of a combination of therapeutic agents, such as a first therapeutic agent for reducing dermal wound scar formation and a second therapeutic agent for increasing wound healing. For example, the second therapeutic agent may be at least one wound healing agent selected from vascular endothelial growth factor, epidermal growth factor, and/or at least one cytokine effective in promoting or improving wound healing, such as IL-7 or IL-10. The second therapeutic agent, may be used in combination with the first therapeutic agent, TGF-β. The therapeutic agents may be administered in any suitable manner known in the art. For example, the first and second scar formation reduction therapeutic agents may be administered sequentially (at different times) or concurrently (at the same time). For example, a first therapeutic agent for scar formation reduction and a second therapeutic agent for increasing wound healing may be administered sequentially (at different times) or concurrently (at the same time). In some embodiments, the first and second therapeutic agents are administered in a separate composition. In some embodiments, the first and second therapeutic agents are in the same composition.

Embodiments of the disclosure relate to compositions and methods comprising therapeutic compositions. The different therapies may be administered in one composition or in more than one composition, such as two, three, four, or more compositions. Various combinations of the agents may be employed.

The therapeutic agents of the disclosure may be administered by the same route of administration or by different routes of administration. In some embodiments, the second therapeutic agent is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized by the body to a metabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

Certain aspects of the present invention also concern kits containing compositions of the invention or compositions to implement methods of the invention. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

It is contemplated that any method or composition described herein can be implemented with respect to any other method or composition described herein and that different embodiments may be combined. The claims originally filed are contemplated to cover claims that are multiply dependent on any filed claim or combination of filed claims.

### EXAMPLES

The following examples are included to demonstrate preferred embodiments of the disclosure. It should be appreciated by those of skill in the art that the techniques disclosed in the examples which follow represent techniques discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—In Situ-Formed Timed-Pulsatile-Release Dressing for Reducing Scar Hypertrophy

Scarring often occurs following deep trauma, severe burn injury, or surgical incision. It can have a profound impact on the quality-of-life of patients, and pose a great challenge to physicians. One method to overcome scarring is to inhibit collagen synthesis by blocking transforming growth factor-β (TGF-β). However, the timing of TGF-β inhibition is crucial, as blocking the signal prematurely will result in poor wound healing, while blocking after peak collagen synthesis will have limited scar-reduction effect. Hence, the inventors developed a novel composite wound photogelation dressing for timed discharge of at least one TGF-β inhibitor in vivo. Specifically, the inventors designed a timed delay-release drug delivery capsule system with poly(lactic-co-glycolic acid) o-nitrobenzene derivative (PLGA-NB) through water-in-oil-in-water (W/O/W) emulsion evaporation method. An efficient phototriggered-imine-crosslinking reaction was then used to integrate the capsule into the wound tissue via hyaluronic acid o-nitrobenzene derivative (HA-NB/HA-CDH). This facile treatment, termed the in situ-formed timed-pulsatile-release dressing (ISTD), can effectively decrease fibroblast activity and collagen deposition at a precise time point, resulting in significance reduction in scar formation in both mouse and rabbit models. In some aspects, the timed delay-release drug delivery capsule system discharges one or more TGF-β inhibitors.

### A. Introduction

Excluding very minor lesions, almost all wounds will lead to scarring of different degrees. Generally, skin wounds that heal within about two weeks with minimal collagen deposition will not form scars. But if the repair process takes longer than three to four weeks, various scars such as stretch marks, keloids, hypertrophic scars or atrophic scars can occur depending on the situations. Scars in the skin usually have inferior function compared to normal skin. For example, they don't have sweat glands and hair follicles, and they may cause symptoms of pruritus and pain.¹ In addition, scars can sometimes restrict range of motion, and present as a permanently visible reminder of the traumatic event.²

Past research on scar models have led to a profound understanding in the pathophysiology of scar formation. One of the main regulators of scarring is TGF-β, which is an attractive target for scar-reduction strategies since there are readily available inhibitors against TGF-β. However, early attempts at targeting TGF-β are unsuccessful as they disregard the critical timing issue of drug deliver. Indeed, Mustoe et, al. have demonstrated that starting anti-TGF-β antibody treatments in the first week after wounding actually delays wound healing without reducing scarring, as TGF-β is required for early wound healing. In contrast, scar formation is reduced when TGF-β inhibition is initiated one week after wounding. Thus there is a clinical need to create a dressing material that allows for pulsatile release of TGF-β inhibitors one week after application. Many other methods such as coating, multiple-compaction have been invented.⁴⁻⁸ While these methods may potentially be utilized for delivering one or more TGF-β inhibitors at an optimal time point to reduce scarring, they have not been validated for this purpose. Moreover, they require sophisticated three-dimensional microtechnology equipment that limits its potential for clinical application.

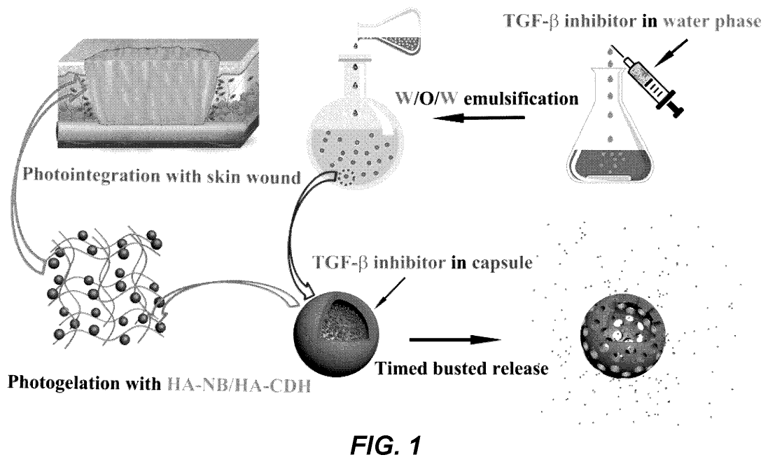

Herein, the inventors demonstrate a novel drug delivery system, the in situ-formed timed-pulsatile-release dressing (ISTD), which can be integrated into the wound via in situ photo triggered-imine-crosslinking pre-gelling polymer to deliver a TGF-β inhibitor at an optimal time point and reduce scarring (FIG. 1). Emulsion-based preparation method is used to load the TGF-β inhibitor inside polymer capsules.⁹⁻¹¹ External 365 nm light irradiation, which is safe and has been widely used in clinical regenerative medicine, is then given to allow the capsules and pre-gelling polymers to quickly crosslink with the surrounding tissue surfaces through o-nitrobenzene and —NH₂ reaction.¹² This mingled cross-linking method stabilizes the timed release capsules to confer even microdopant packing, leading to trailed cargo discharge in the wound area, which may enhance the therapeutic effect.¹³ Finally, the timed-degradation of the polymer barrier results in the pulsatile release of the TGF-β inhibitor to achieve optimal scar reduction potential. The ISTD shows excellent timed controllability, biocompatibility and efficient tissue integration. Most importantly, the system can significantly reduce hypertrophic scar formation without delaying wound healing in both mouse and rabbit skin wounding models. As discussed above, the drug delivery system can be configured to release two or more TGF-β inhibitors.

### B. Materials and Methods

1. Materials

PLGA-NB (75:25; MW 50,000) and HA-NB/HA-CDH were received as a kind gift from Linyong Zhu's lab at East China University of Science and Technology.¹² TGF-β inhibitor SB431542 is purchased from selleckchem, Inc. Polyvinyl alcohol (PVA; MW 95,000) and poly(ethylene glycol) (PEG; MW 400) were obtained from Sigma-Aldrich, Inc. Milli-Q185 water (Waters, Saint-Quentinen-Yveline, France) was used in all experiments. The fluorescent dyes, 1,1′-dioctadecyl-3,3,3′,3′-tetramethylindocarbocyanine perchlorate (DiI) and 1,1′-dioctadecyl-3,3,3′,3′-tetramethylindocarbocyanine perchlorate (DiD), were obtained from Fisher Scientific, Inc. All other chemicals were of analytical grade and obtained from Sigma-Aldrich, Inc.

2. Timed Release Capsule Fabrication

All capsules were produced by W/O/W emulsion method followed by solvent evaporation technique. The inventors first prepare 50 μL deionized water with 2.5 mg PEG to get the inner water phase. After 15 minutes of sonication, this solution was droplet added into a solution of 100 mg PLGA-NB dissolved in 1 mL of chloroform with vigorously stirring. This process was performed over at least 10 minutes to allow for emulsification. The resulting water-in-oil (W/O) emulsion was then transferred into a 15 mL aqueous solution composed of 1% (wt/vol) PVA used as an emulsion stabilizer. The double emulsion (W/O/W) was obtained using 3000 rpm stirring by a magnetic stirrer (C-MAG HS 7; IKA Works, Inc.), and then placed in a laboratory fume hood for 6 hours for solvent evaporation. The capsules with a blank core (Cap-Hollow) recovered after centrifugation was wash by deionized water and then lyophilizes using the freeze dryer (Virtis Benchtop; SP Industries, Inc.).

Capsules with timed release TGF-β inhibitor (Cap-I) was produced by the same method, except when during the preparation of the inner water phase, 5 mg TGF-β inhibitor was added to the deionized water and PEG. Similarly, to create the bovine serum albumin (BSA)-filled capsules (Cap-BSA), 5 mg BSA was dispersed in deionized water and PEG when generating the inner water phase. Likewise, for the timed release capsules containing förster resonance energy transfer (FRET) fluorophores (Cap-FRET), DiI and DiD (2.5 nmol of DiI; DiD:DiI 4:1) were dissolved in ionized water and PEG for the inner water phase.

Particle size, surface morphology and inner hollow structure were determined by a light microscope (EVOS FL; Advanced Microscopy Group) and a high-resolution Field-Emission Scanning Electron Microscope (FESEM; Merlin; Carl Zeiss, Inc.). For light microscope, the capsules were freeze-dried and re-dispersed onto a glass slide prior to imaging. For FESEM, the dispersed PLGA-NB capsule in water was dropped onto a silica chip and air dried. The silica chip with PLGA-NB particles was coated with platinum at 20 mA for 70 s under vacuum. The images were taken using FESEM at an acceleration voltage of 2 kV.

3. Cytotoxicity Evaluation of the Capsules

The Cytotoxicity of human colorectal carcinoma (Caco-2) cells and human epithelial carcinoma (HeLa) cells was assessed by the Cell Counting Kit-8 (CCK-8) method. This colorimetric cell proliferation kit allows for easy and reliable colorimetric determination of viable cell numbers with excellent sensitivity and linearity. Caco-2 cells and HeLa cells were purchased from Highveld Biologicals, Johannesburg, South Africa. Stocks of the cells were prepared in culture medium containing 80% (vol/vol) fetal bovine serum and 10% (vol/vol) dimethyl sulfoxide and kept in liquid nitrogen until further use. The cells were maintained according to routine cell culture procedures. To determine cell viability after exposure of all cell lines to different concentrations incubated for 24 hours, the CCK-8 assay was performed according to manufacturer's instructions. Briefly, the cells were seeded in 96-well plates at 37° C. in humidified atmosphere (90% humidity), 5% CO2, Dulbecco's minimal essential medium, 1% (wt/vol) nonessential amino acids, 1% (wt/vol) glutamine, 10% (vol/vol) fetal bovine serum, penicillin, (100 U/mL), and streptomycin (100 μg/mL). When 50% confluence was reached, the tested capsules were dispersed in cell growth medium at 1, 10, and 100 μg/mL and added to the wells. After incubating for 24 hours, the medium was removed, and after washing the cells with PBS, CCK-8 reagent was added to each well followed by 2 hours of incubation at 37° C. The absorbance was measured using a microplate reader (Synergy Neo; BioTek Instruments, Inc.) at 450 nm.

4. In Vitro Release Kinetics of Timed Release Capsule

Cap-BSA was placed into 5 mL phosphate-buffered saline (PBS) in a centrifuge tube and incubated on a shaker at 37° C. Release was then measured everyday using a Pierce™ BCA Protein Assay Kit (Thermo Fisher Scientific Inc.). Results were quantified using a standard curve and normalized to total cumulative release (n=5). At each time point, supernatant was replaced with 5 mL of fresh PBS. The timing of release was reported as the day when more than 50% of the total payload had been released.

5. In Vivo Release Kinetic of Timed Release Capsule

WT CD1 mice were obtained from the Transgenic Core Facility at University of Chicago. All the mice were housed under pathogen-free conditions in ARC (animal resource center) of University of Chicago under a 12 hours light-dark cycle. All the subjects were not involved in any previous procedures. The experimental protocol was reviewed and approved by the Institutional Animal Care and Use Committee of University of Chicago. Cap-FRET was used in vivo kinetics in 3 CD1 mice. Cap-FRET was sterilized prior to surgery using a 20 μl drop of 70% ethanol. Before injection, mice were anesthetized using continuous inhalation of 3% isoflurane and had their injection site sterilized with ethanol. 30 mg capsule was injected subcutaneously into the dorsum of each animal.

Mice were imaged using an in vivo imaging system (IVIS 200; Xenogen Corporation) daily. At each imaging session, mice were anesthetized using continuous inhalation of 3% isoflurane and placed on the heated imaging platform. Fluorescent images were then collected using 560/620 nm excitation/emission filter sets with an 1.00 second exposure time, F-Stop setting of 1, medium binning and subject height of 1.5 cm. Cumulative release was assigned to the maximum and minimum overall fluorescence in the region of interest match a particular capsule's release and background signal, respectively. Release timing was reported as the day at which the max radiant efficiency increased one fold in the corresponding.

6. Mouse Skin Wound Model and Treatment

Eighteen CD1 male mice aged 6-8 weeks were used in this study. The mice were anesthetized using continuous inhalation of 3% isoflurane. After shaving, 6 wounds (6 mm in diameter) were created on the dorsal side of each mouse by removing full-thickness skin via 6-mm punch biopsy. Nine of the mice received ISTD on 3 of the 6 wounds (Cap-I group), and hollow capsules (Cap-Hollow group) on the other 3. After applying the dressing, the material was placed under 365 nm LED light (20 mW/cm) for 3 minutes to activate the crosslinking reaction. The other nine mice were treated with normal saline for all 6 wounds (Control saline group). Finally, Tegaderm™ films (3M Inc.) were used to cover the wounds and prevent water loss in all the mice until the wounds are fully epithelialized.

At day 5, 10 and 15 post-surgery, 3 mice in each group were euthanized and the wounded skin removed, fixed in formalin, embedded in paraffin, and sectioned. Hematoxylin and eosin (H&E), trichrome, F4/80 antibody, anti-alpha smooth muscle actin antibody (α-SMA), CD4 antibody and CD8 antibody staining was used for histological observations. Antibodies were diluted according to manufacturer's instruction, unless indicated otherwise. The sections with F4/80 staining were observed under the microscope (Eclipse Ti2; Nikon Inc.) at 200× magnification while the sections with anti-alpha smooth muscle actin (α-SMA), CD4 and CD8 staining were observed at 400× magnification. Five fields were randomly selected from each section to count the F4/80 positive macrophages, α-SMA positive fibroblast cell, CD4 and CD8 positive T-cells. Immunoreactive cells were quantified as the mean cell count expressing the appropriate positive marker per high-power field (HPF). The severity of dermal fibrosis was scored on 0 to 6 scales by combining the extent (0-3 score) and density (0-3 score) of collagen deposition based on trichrome stain. The values were measured twice by two blinder examiners and then averaged. Histological data are expressed as mean±standard deviation (SD). Statistical analysis was performed by a paired two-tailed Student's t-test. A p value of <0.05 was considered significant.

7. Rabbit Ear Hypertrophic Scar Model and Treatment

The inventors utilized a reproducible and quantifiable dermal ulcer model proposed by Mustoe et al.¹⁴ An adult New Zealand White female rabbit was used for this model. The rabbit was anesthetized with an intramuscular injection using ketamine (60 mg/kg) and xylazine (5 mg/kg). Six wounds were created on the ventral side of right ear using 7-mm dermal biopsy punch to reach the cartilage. The cartilage was meticulously notched without full dissection, while epidermis, dermis, and perichondrium are scrupulously removed using a dissecting microscope. This process would delay epithelialization and increase the degree of hypertrophic scaring, which leads to persistent scar elevation.¹⁵ Two of the 6 wounds were either assigned to the Cap-I group: wounds with ISTD treatment (treated with fully mixed 50 μL 2.5% wt HA-NB/HA-CDH and 3 mg Cap-I); Cap-Hollow group: wounds treated with fully mixed 50 μL 2.5% wt HA-NB/HA-CDH and 3 mg Cap-Hollow; or the Control saline group: wounds are treated with saline. After materials were applied, the wounds were placed under a 365 nm LED light (20 mW/cm) for 3 minutes to activate crosslinking. Finally, Tegaderm™ films were applied to cover the wounds until the wounds are fully epithelialized. After 30 days, a second procedure was performed on the left ear with the same process, except that instead of 6 wounds, 12 wounds were created, with four wounds in each groups. Finally, 30 days after the surgery on the left ear, the rabbit was euthanized and the ears collected. The wounds were resected, fixed in formalin and embedded in paraffin. Sections were made across the most elevated portion of the scar and stained with H&E and trichrome. For histomorphometric analysis, the scar elevation index (SEI) which measures the ratio of total scar connective tissue area to the area of underlying dermis is assessed.¹⁵ The thickness of the dermis is determined based on the adjacent unwounded dermis. Histological data are expressed as mean±standard deviation (SD). Statistical analysis was performed by a paired two-tailed Student's t-test. A p value of <0.05 was considered significant.

### C. Results and Discussion

1. Capsule Fabrication and Cytotoxicity

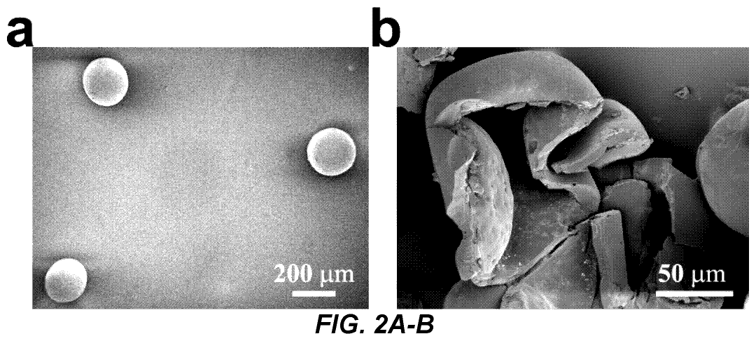

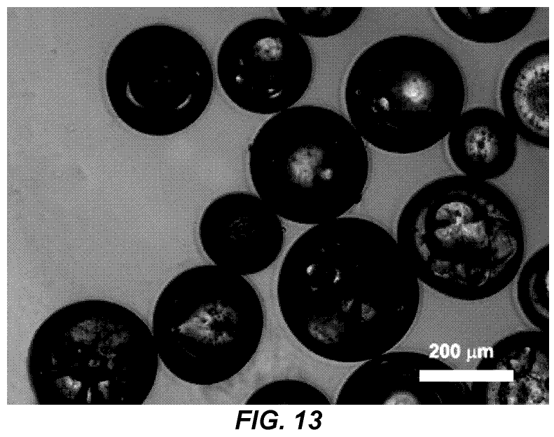

A typical method to obtain a biphasic pulsatile release profile is to establish a core-shell vehicle. In this vehicle, an active cargo is encapsulated in a water-insoluble biodegradable polymer shell to enable a delayed pulsatile release. For many years, drugs such as vaccine and antigen have been encapsulated inside biodegradable capsules via the W/O/W double emulsion method.¹⁶ FESEM was used to assess the size and structure of the capsules. As shown in FIG. 2a, the capsules are well-defined spherical particles with smooth surface without pores or cavities. The inventors next crushed the Cap-Hollow capsules and examined them under FESEM to examine the internal structure of the capsules (FIG. 2b). The capsule contains a hollow inner structure with a single-core. The aqueous droplets that were entrapped in the interior of the capsules evaporated during the drying process and formed semi-sphere pits on the inner wall. The size of the capsule was determined by both light microscope (FIG. 13) and FESEM. The number-averaged diameter of the capsules was 220±20 μm and the thickness of the wall was approximately 20±3 μm. The W/O/W emulsion method followed by solvent evaporation technique enables the fabrication of classic hollow biodegradable polymer capsules, which have previously been shown to allow various controllability based on their physical and chemical properties, especially with materials such as PLGA and its derivatives.¹⁷⁻²⁰ PLGA is a biodegradable and biocompatible copolymer that has been used in many drug products approved by the Food and Drug Administration.^{21,22} By changing the lactic/glycolic acid ratio, molecular weight or the end group of the copolymer, physicochemical characteristics of the polymer, such as glass transition temperature and degradation rate can be tailored to suite specific needs.²³ The timed controllability of biodegradation rate of PLGA and its derivatives provides an ideal platform to create a micro-vehicle for timed pulsatile delivery.^{3,24} Additionally, the capsules the inventors prepared are sufficiently suitable for biomedical applications since they are small enough to be blended into wound dressing.

2. Release Kinetics of Times Release Capsule

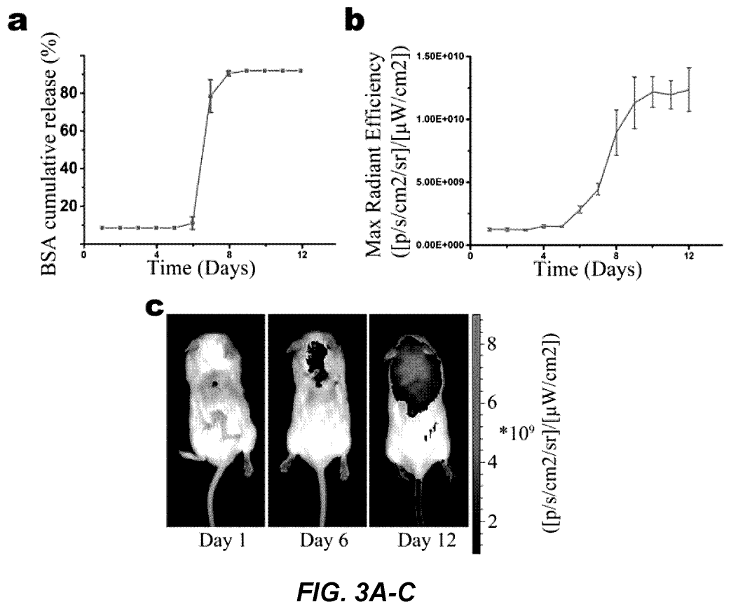

Based on previous reports, the inventors were able to design a polymer with specific composition and degradation schedule, thus creating a PLGA derivative capsule that can burst at a predefined time window.^{3,25} The inventors first examined the pulsatile release profile of the capsule in vitro by incubating Cap-BSA capsules in PBS and measuring the accumulative BSA level. As shown in FIG. 3a, BSA level remained undetectable for 6 days, but dramatically increased during day 6-8. This indicates that the capsule content is indeed released in pulse, and there is minimal leakage prior to PLGA-NB degradation.

The inventors next examined the in vivo release kinetics of the capsules by injecting Cap-FRET capsules into mice subcutaneously, and monitoring the change in fluorescence level using IVIS. IVIS is a relatively recent, real-time and noninvasive imaging technology, which benefits the tracking the fate of inorganic or organic drug carriers. FRET is a nonradiative energy transfer process and often used as a “nanoscale ruler”. It relied on the interactions between donor and acceptor fluorophores in spatial proximity (within 2-10 nm distance). When the donor fluorophore emission spectrally overlaps with the acceptor absorption, a decrease of the fluorescence lifetime and quantum yield of the donor in the presence of the acceptor will occur. Notably, this interaction declines exponentially with increasing donor-acceptor distance R (FRET efficiency EFRET∝1/R6). FRET imaging technique has been applied to solve various biological issues such as the detection enzyme activity in living cell, protein location, measurement of distance among molecular interactions and lipid membrane dynamics.²⁶⁻³⁰ In this study, the inventors utilized DiI/DiD FRET pair loaded capsule with an optimized fluorophore ratio to monitor the release kinetic in vivo. DiI and DiD were designed for cell membrane labeling at first, and they have strong spectral overlap between DiI emission and DiD absorption (Förster radius R0=5.2 nm), which leads to efficient interaction of FRET when they are encapsulated together.31 The donor is DiI which has excitation at 535 nm and emission at 640 nm (±10 nm). The acceptor is DiD which has excitation at 633 nm and emission at 680 nm (±10 nm). So the FRET was defined as using the 535 nm excitation and emission at 680 nm (±10 nm). It has been proven that FRET interactions between donors and acceptors will be disrupted when the coencapsulation is destroyed.³² In this study, when the polymer capsule is cracked, which results in the dispersion and separation of DiI/DiD FRET pair in mice, DiI fluorescence will no longer be transferred to DiD, and its emission will resume.³³ As seen by FIG. 2b acquired in the DiI channel (560/620 nm excitation/emission filter set in IVIS, which were found to be the most effective filter set that provided a high signal while minimizing fluorescence overlap and hindrance from skin tissue and hair), a similar trend with in vitro BSA release from Cap-BSA was observed when Cap-FRET were subcutaneously injected into mice. Cap-FRET discharged its payload after around 6 days, as indicated by a ˜8 fold increase in fluorescence upon release. Graphs in FIG. 2c shows corresponding images of mouse collected with IVIS. During the first 5 days after subcutaneously injection, minimal fluorescent signal was observed, indicating that the Cap-FRET capsules remained intact. A sharp increase in signal was then observed starting at day 6. This is in agreement with the in vitro experiment, and indicates that the PLGA-NB shell starts to degrade between day 6-8, resulting in the release of DiI and DiD into surrounding tissue.

3. Mouse Skin Wound Dressing

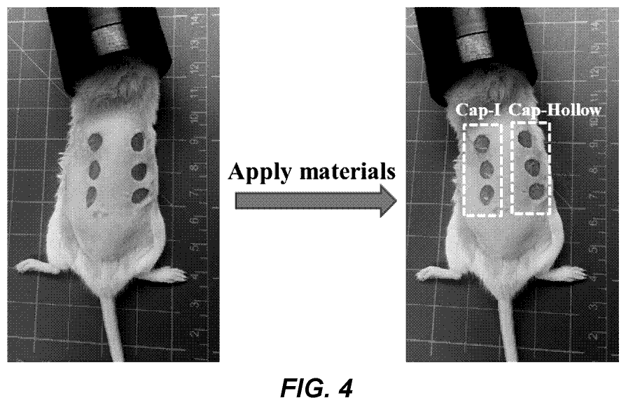

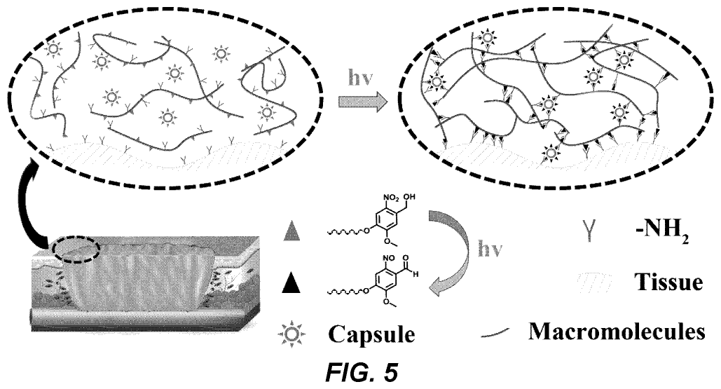

Having determined that the capsules are biocompatible and has an accurate delayed pulse release schedule suitable for timed TGF-β release, the inventors proceeded to evaluate the applicability of the system in vivo using mouse skin wounding model. First, 6 open wounds were created on the dorsal skin on each mouse suing a 6-mm biopsy punch. Next, Cap-I capsules were applied to the skin wound in a HA-NB/HA-CDH hydrogel (FIG. 4). The hydrogel has been shown to be biocompatible and has efficient photogelation capacity.12 A unique photo-triggered crosslinking reaction was used to integrate HA-NB/HA-CDH with the PLGA-NB polymer shell of the capsules and with the wound tissue (FIG. 5). During the reaction, the o-nitrosobenzaldehyde groups are converted by o-nitrobenzene upon 365 nm illumination, and quickly crosslink with —NH₂ in the polymers and surrounding wound surfaces.12,34 It not only offers excellent spatiotemporal controllability for quick and easy application in clinics but also provided facile biocompatibility and tissue adhesion simultaneously.³⁵⁻³⁷

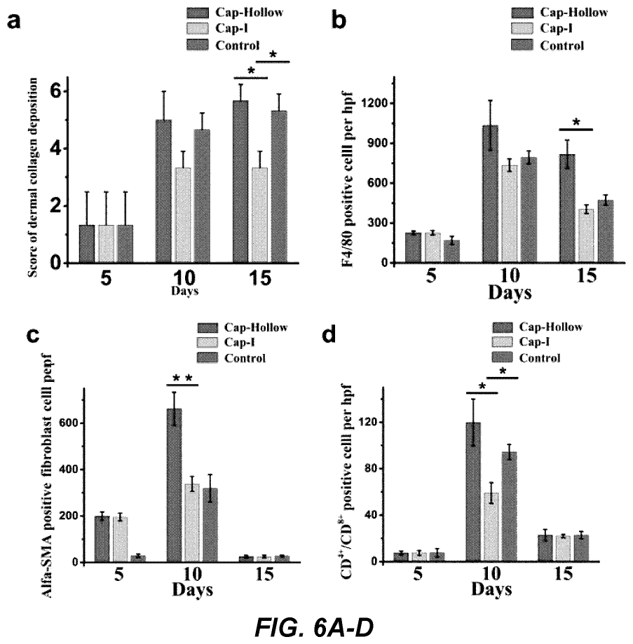

After 5, 10, and 15 days post-surgery, the skin was sectioned, stained with H&E, trichrome, and various antibodies for further evaluation. Based on the histological sections, skin from the Cap-I group shows less fibrosis at day 15 compared to the Cap-Hollow or Control groups. Importantly, the Cap-I group did not show delayed in wound healing at day 5, indicating the absence of TGF-β leakage during early wound healing phase. Tricrhome stain was used to highlight collagen fibers and to quantify the severity of fibrosis using a designated scoring scheme: the extent of fibrosis was first assessed and given 0-3 score (0 for no fibrosis and 3 for fibrosis involving full skin thickness), then the density of collagen fibers were assessed and given another 0-3 score (0 for loose collagen fibers similar to normal skin and 3 for highly dense collagen fibers), and finally the two scores are combined to get the fibrosis score (0 for no fibrosis, 1-2 for mild fibrosis, 3-4 for moderate fibrosis, and 5-6 for severe fibrosis). As shows in FIG. 6a, the fibrosis score was similar between Cap-hollow, Cap-I and Saline treated skin five days after surgery. However, after 15 days, the fibrosis score was significantly lower in the Cap-I group (3.3±0.6) compared to Cap-hollow (5.7±0.6, p<0.05) or Control groups (5.3±0.6, p<0.05). These results demonstrated that timed delay TGF-β inhibitor release from Cap-I dressing significantly reduced fibrosis in the wounded tissue.

To evaluate the effect of TGF-β inhibition at the tissue level, immunohistochemistry was performed on the tissue sections. F4/80 antibody was used to highlight tissue macrophages which are indicators of wound repair and fibrosis. Activated fibroblasts can be positively identified using the specific α-SMA marker. Finally, CD4 and CD8 antibodies are used highlight the two T-cell populations associated with tissue healing and cytotoxic reaction, respectively. Consistent with the findings under H&E and trichrome stain, the Cap-I group showed significantly lower number of macrophages, activated fibroblasts, and CD4 cells compared to Cap-hollow group at 10 days post-surgery (FIGS. 6b-d). There were very few CD8+ T-cells in all of the sections (0-1/10HPF, data not shown). However, activated fibroblast and CD4+ T-cell number were similar across all three groups at day 15 (FIGS. 6c-d). This may be due to the general lack of fibrosis activity in mouse skin during wound healing, as the wound can be recovered via muscle contraction. Overall, in vivo evaluation using mouse model supports the use of ITSD in reducing scar formation.

4. Rabbit Ear Scar Model

As mouse skin has limited ability to form scar tissue following wounding, the inventors attempt to replicate their success in a more clinically relevant model by utilizing a reproducible and quantifiable rabbit ear model. This dermal ulcer model was first described by Joseph and Dyson in 1966 and subsequently developed by Morris.³⁸ Because the rabbit ear is covered by tense skin with lower hair density, it highly resembles human skin, and provides several advantages as a scar model. First, the wound does not heal via muscle contraction, so epithelialization is delayed and a raised scar can form.¹⁵ In addition, the scar tissue also parallels that of human in terms of morphology and therapeutic responsiveness, and can be assessed grossly as well as microscopically.

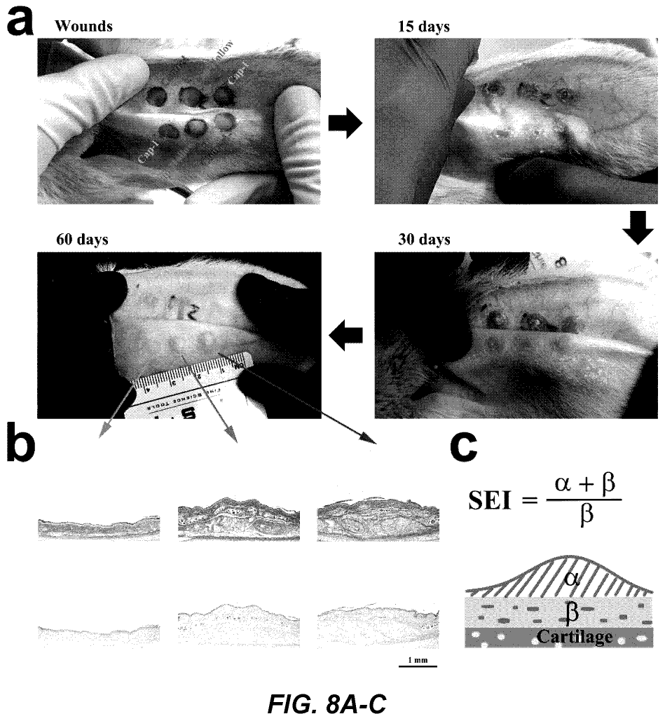

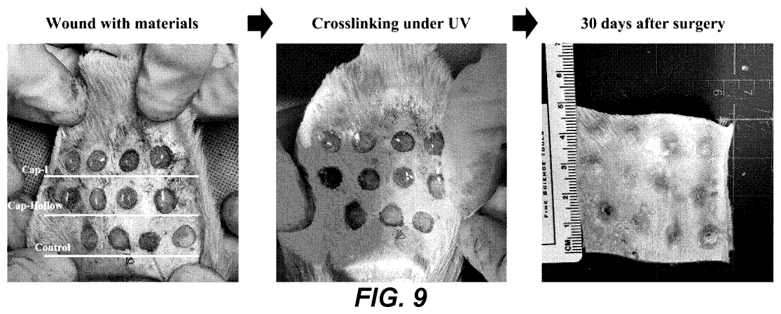

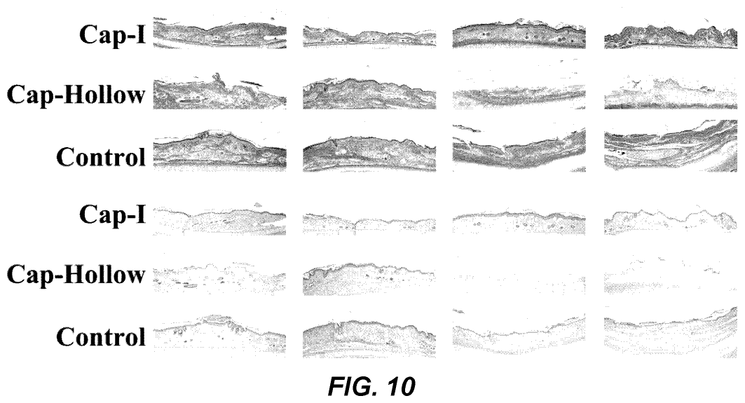

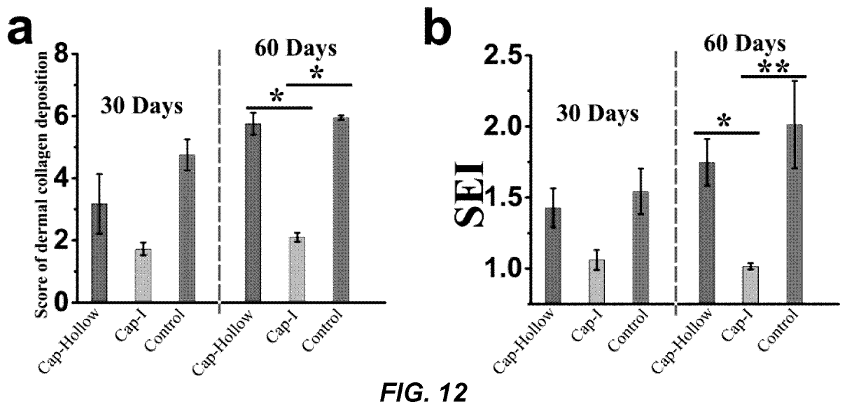

As indicated in FIG. 8a, the inventors treated the ear with reverse treatment sequences to minimize possible deviation introduced by differences in wound location (proximal versus distal ear). The wounds healed normally in all groups, and were fully epithelialized at 15 days post-surgery, and the difference between the three groups can be observed grossly during day 15-30 post-surgery (FIG. 8a, FIG. 9). Interestingly, Cap-I and Cap-Hollow groups also showed faster epithelial ingrowth grossly compared to the Control group, indicating that photo-gelation dressing alone could help promote wound healing. Histological evaluation of the dermal compartment reveals significant differences in overall cellularity and collagen fiber deposition 30 days after surgery (FIG. 10, 12a). Consistent with previous result obtained from the mouse model, the Cap-I group showed significantly lower fibrosis score (1.7±0.2) compared to Cap-hollow group (3.2±1.0) or Control group (4.8±0.5). The effect of ITSD can also be demonstrated using the robust SEI calculation, (FIG. 12b). Notably, the scar-reducing effect in the Cap-I group is still present over long term, as seen grossly and microscopically 60 days post-surgery (FIG. 8b, 11, 12).

5. Porcine Skin Model

Porcine skin resembles human skin anatomically and physiologically. The full-thickness excisional wounding model in porcine skin is one of the best preclinical wound healing models. To determine the effectiveness of the dermal wound scar reducing methods and compositions in a preclinical setting, healing and scarring of full-thickness cutaneous wounds on the dorsal skin of Yorkshire pigs was examined.

Three male Yorkshire porcine (25-30 kg) were fasted for 12 h before surgery. Briefly, the animal was anesthetized with an injection of ketamine (20 mg/kg, IM), followed by propofol (1 mg/kg, IV), and then intratracheally intubated and ventilated. Anesthesia was maintained with 4 mg kg⁻¹ h⁻¹ propofol during the surgery. The anesthetized pigs had their backs depilated, immobilized and placed in a dorsal position. The dorsal skin was cleaned with water and soap, and sterilized with iodine and 75% alcohol. For the creation of the defect, 12 areas of skin wound were created by removing 3 cm×3 cm of full thickness skin in the central back along the thoracic and lumbar area. Incisions were with a surgical blade to the panniculus carnosus layer and the overlying skin was excised. After application of hydrogel and capsules, the wounds were illuminated with a 365 nm LED light (20 mW/cm) for 3 minutes to activate crosslinking. Large Tegaderm bandages (3M Inc.) were used to cover the wounds and followed by 3M loban 2 antimicrobial drape around the perimeter, forming a watertight dressing, and finally a specially designed jacket to hold the bandage in place. The wounded skin tissues were collected at day 20 and 50 post-surgery. Samples were resected, fixed in formalin and embedded in paraffin, followed by H&E and trichrome staining for histomorphometric observations.

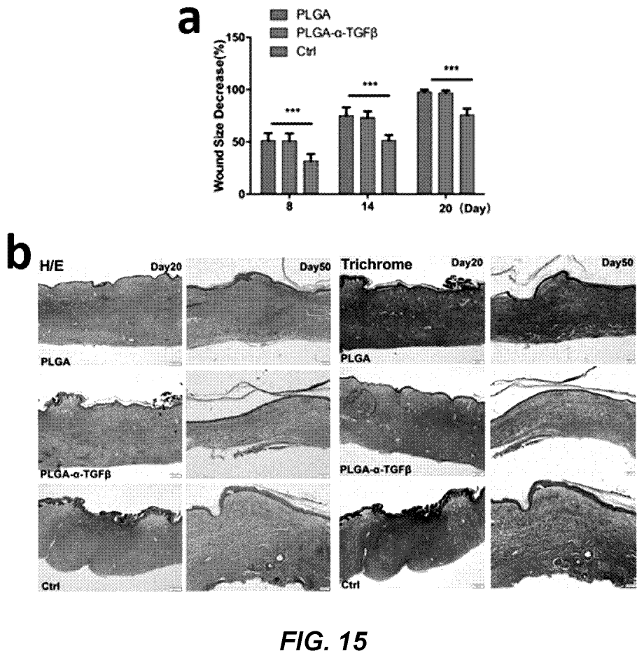

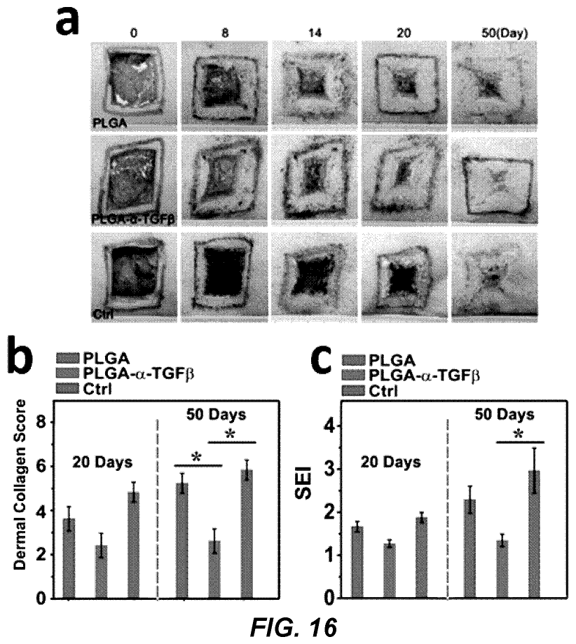

Consistent with the data from rodent skin models, application of the capsules with or without TGFβ inhibitors by photogelation can enhance wound closure (FIGS. 15A and 16A). Trichrome staining indicated significantly less collagen deposition in wounds treated with inhibitor-loaded PLGA-NB capsules (FIGS. 15B and 16B). Histological evaluation also indicated significantly reduced SEI for wounds treated with TGFβ-inhibitor containing capsules (FIG. 16C).

### D. Conclusion

The inventors here report a unique in situ-formed timed-pulsatile-release dressing treatment. With a confirmed controllable timed discharging from micro-size vehicle, the dressing can release its cargo in vitro and in vivo within a predetermined time window. The inventors have shown that the dressing can be facilely implemented to reduce hypertrophic scar formation without compromising wound healing in mouse, rabbit, and porcine models. Overall, the current work takes a significant step toward developing a tissue-integratable, biocompatible and controllable timed-pulsatile-release system that could not only be used in reducing scar formation, but may potentially be applied to wider aspects of tissue engineering and regenerative medicine.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1 Su, C. W., Alizadeh, K., Boddie, A. & Lee, R. C. The problem scar. Clinics in plastic surgery 25, 451-465 (1998).
2 Berman, B. et al. A review of the biologic effects, clinical efficacy, and safety of silicone elastomer sheeting for hypertrophic and keloid scar treatment and management. Dermatologic surgery 33, 1291-1303 (2007).
3 McHugh, K. J. et al. Fabrication of fillable microparticles and other complex 3D microstructures. Science 357, 1138-1142, doi:10.1126/science.aaf7447 (2017).
4 Cleland, J. L. Single-administration vaccines: controlled-release technology to mimic repeated immunizations. Trends in biotechnology 17, 25-29 (1999).
5 De Geest, B. G., De Koker, S., Demeester, J., De Smedt, S. C. & Hennink, W. E. Pulsed in vitro release and in vivo behavior of exploding microcapsules. Journal of Controlled Release 135, 268-273 (2009).
6 Guse, C., Koennings, S., Blunk, T., Siepmann, J. & Göpferich, A. Programmable implants—from pulsatile to controlled release. International journal of pharmaceutics 314, 161-169 (2006).
7 Sungthongjeen, S., Puttipipatkhachorn, S., Paeratakul, O., Dashevsky, A. & Bodmeier, R. Development of pulsatile release tablets with swelling and rupturable layers. Journal of controlled release 95, 147-159 (2004).
8 Tzeng, S. Y. et al. Stabilized single-injection inactivated polio vaccine elicits a strong neutralizing immune response. Proceedings of the National Academy of Sciences 115, E5269-E5278 (2018).
9 Zhang, J. et al. Construction of versatile multilayered composite nanoparticles from a customized nanogel template. Bioactive materials 3, 87-96 (2018).
10 Zhang, J., Yang, F., Shen, H. & Wu, D. Controlled formation of microgels/nanogels from a disulfide-linked core/shell hyperbranched polymer. ACS Macro Letters 1, 1295-1299 (2012).
11 Cui, W. et al. Preparation and evaluation of poly (L-lactide-co-glycolide)(PLGA) microbubbles as a contrast agent for myocardial contrast echocardiography. Journal of Biomedical Materials Research Part B: Applied Biomaterials: An Official Journal of The Society for Biomaterials, The Japanese Society for Biomaterials, and The Australian Society for Biomaterials and the Korean Society for Biomaterials 73, 171-178 (2005).
12 Yang, Y. et al. Tissue-Integratable and Biocompatible Photogelation by the Imine Crosslinking Reaction. Advanced Materials 28, 2724-2730 (2016).
13 Zhang, J. et al. Ionic colloidal molding as a biomimetic scaffolding strategy for uniform bone tissue regeneration. Advanced Materials 29, 1605546 (2017).
14 Kloeters, O., Tandara, A. & Mustoe, T. A. Hypertrophic scar model in the rabbit ear: a reproducible model for studying scar tissue behavior with new observations on silicone gel sheeting for scar reduction. Wound Repair and Regeneration 15, S40-S45 (2007).
15 Morris, D. et al. Acute and chronic animal models for excessive dermal scarring: quantitative studies. Plastic and reconstructive surgery 100, 674-681 (1997).
16 Alonso, M. J. et al. Determinants of release rate of tetanus vaccine from polyester microspheres. Pharmaceutical research 10, 945-953 (1993).
17 Sanna, V. et al. Effect of chitosan concentration on PLGA microcapsules for controlled release and stability of resveratrol. International journal of biological macromolecules 72, 531-536 (2015).
18 Abulateefeh, S. R. & Alkilany, A. M. Synthesis and characterization of PLGA shell microcapsules containing aqueous cores prepared by internal phase separation. AAPS PharmSciTech 17, 891-897 (2016).
19 Nomura, T. & Routh, A. F. Benign preparation of aqueous core poly lactic-co-glycolic acid (PLGA) microcapsules. Journal of colloid and interface science 513, 1-9 (2018).
20 Tang, S. et al. Imaging-guided synergetic therapy of orthotopic transplantation tumor by superselectively arterial administration of microwave-induced microcapsules. Biomaterials 133, 144-153 (2017).
21 Fredenberg, S., Wahlgren, M., Reslow, M. & Axelsson, A. The mechanisms of drug release in poly (lactic-co-glycolic acid)-based drug delivery systems—a review. International journal of pharmaceutics 415, 34-52 (2011).
22 Makadia, H. K. & Siegel, S. J. Poly lactic-co-glycolic acid (PLGA) as biodegradable controlled drug delivery carrier. Polymers 3, 1377-1397 (2011).
23 Xu, Y., Kim, C. S., Saylor, D. M. & Koo, D. Polymer degradation and drug delivery in PLGA-based drug-polymer applications: A review of experiments and theories. Journal of Biomedical Materials Research Part B: Applied Biomaterials 105, 1692-1716 (2017).
24 Beugeling, M. et al. The mechanism behind the biphasic pulsatile drug release from physically mixed poly (dl-lactic (-co-glycolic) acid)-based compacts. International journal of pharmaceutics 551, 195-202 (2018).
25 Siegel, S. J. et al. Effect of drug type on the degradation rate of PLGA matrices. European journal of pharmaceutics and biopharmaceutics: official journal of Arbeitsgemeinschaft fur Pharmazeutische Verfahrenstechnik e.V 64, 287-293, doi:10.1016/j.ejpb.2006.06.009 (2006).
26 Jares-Erijman, E. A. & Jovin, T. M. FRET imaging. Nature biotechnology 21, 1387 (2003).
27 Sekar, R. B. & Periasamy, A. Fluorescence resonance energy transfer (FRET) microscopy imaging of live cell protein localizations. The Journal of cell biology 160, 629-633 (2003).
28 Vaknin, A. & Berg, H. C. Single-cell FRET imaging of phosphatase activity in the Escherichia coli chemotaxis system. Proceedings of the National Academy of Sciences 101, 17072-17077 (2004).
29 Fruhwirth, G. O. et al. How Förster resonance energy transfer imaging improves the understanding of protein interaction networks in cancer biology. Chemphyschem 12, 442-461 (2011).
30 Sahoo, H. Förster resonance energy transfer—A spectroscopic nanoruler: Principle and applications. Journal of Photochemistry and Photobiology C: Photochemistry Reviews 12, 20-30 (2011).
31 Roger, E., Gimel, J.-C., Bensley, C., Klymchenko, A. S. & Benoit, J.-P. Lipid nanocapsules maintain full integrity after crossing a human intestinal epithelium model. Journal of Controlled Release 253, 11-18 (2017).
32 Gravier, J. et al. FRET imaging approaches for in vitro and in vivo characterization of synthetic lipid nanoparticles. Molecular pharmaceutics 11, 3133-3144 (2014).
33 Chen, H. et al. Fast release of lipophilic agents from circulating PEG-PDLLA micelles revealed by in vivo forster resonance energy transfer imaging. Langmuir 24, 5213-5217 (2008).
34 Pauloehrl, T. et al. (Bio) molecular surface patterning by phototriggered oxime ligation. Angewandte Chemie International Edition 51, 9181-9184 (2012).
35 DeForest, C. A., Polizzotti, B. D. & Anseth, K. S. Sequential click reactions for synthesizing and patterning three-dimensional cell microenvironments. Nature materials 8, 659 (2009).
36 Liu, Z., Liu, T., Lin, Q., Bao, C. & Zhu, L. Sequential control over thiol click chemistry by a reversibly photoactivated thiol mechanism of spirothiopyran. Angewandte Chemie International Edition 54, 174-178 (2015).
37 Liu, Z. et al. Spatiotemporally Controllable and Cytocompatible Approach Builds 3D Cell Culture Matrix by Photo-Uncaged-Thiol Michael Addition Reaction. Advanced Materials 26, 3912-3917 (2014).
38 Joseph, J. & Dyson, M. Tissue replacement in the rabbit's ear. British Journal of Surgery 53, 372-380 (1966).
39 Lahn, M. et al. TGF-β inhibitors for the treatment of cancer. Expert Opinion on Investigational Drugs 14, 629-643 (2005).
40 Yingling, J. M. et al. Development of TGF-β signalling inhibitors for cancer therapy. Nature Reviews Drug Discovery 3, 1011-1022 (2004).

1. A method for reducing dermal wound scar formation in a subject in need thereof, the method comprising:
administering to the wound a composition comprising at least one TGF-β inhibitor and a crosslinkable polymeric composition; and
inducing crosslinking of the crosslinkable polymeric composition to form a crosslinked polymeric composition, wherein crosslinking the crosslinkable polymeric composition covalently attaches the crosslinked polymeric composition to dermal tissue.

2. The method of claim 1, wherein the at least one TGF-β inhibitor is provided in a delayed release capsule that is formulated to delay release of the at least one TGF-β inhibitor for a period ranging from one day to sixty days.

3. The method of claim 1 or 2, wherein the delayed release capsule comprises an o-nitrobenzyl functionalized polymer.

4. The method of any of claims 1 to 3, wherein the o-nitrobenzyl functionalized polymer is o-nitrobenzyl functionalized poly(lactic acid).

5. The method of any of claims 1 to 4, wherein the o-nitrobenzyl functionalized polymer is o-nitrobenzyl functionalized is poly(glycolic acid).

6. The method of any of claims 1 to 5, wherein the o-nitrobenzyl functionalized polymer is o-nitrobenzyl functionalized poly(lactic-co-glycolic acid) (PLGA).

7. The method of any of claims 1 to 6, wherein the delayed release capsule comprises polyvinyl alcohol (PVA).

8. The method of any of claims 1 to 7, wherein the delayed release capsule comprises polyethylene glycol (PEG).

9. The method of any of claims 1 to 8, wherein the at least one TGF-β inhibitor is selected from the group consisting of SB431542, LDN-193189, galunisertib (LY2157299), LY2109761, SB525334, LY 3200882, SB505124, pirfenidone, GW788388, LY364947, RepSox, LDN-193189, K02288, SD-208, LDN-214117, SIS3, vactosertib (TEW-7197), DMH1, LDN-212854, ML347, kartogenin, hesperetin, alantolactone, GC-1008, and LY550410.

10. The method of any of claims 1 to 9, wherein the crosslinkable polymeric composition comprises an o-nitrobenzyl functionalized polysaccharide, polypeptide, protein, or hydrophilic or water-soluble polymer.

11. The method of any of claims 1 to 10, wherein the crosslinkable polymeric composition further comprises a terminal amine-functionalized polysaccharide, polypeptide, protein, or hydrophilic or water-soluble polymer.

12. The method of any of claims 1 to 11, wherein the o-nitrobenzyl functionalized polysaccharide is o-nitrobenzyl functionalized hyaluronic acid.

13. The method of any of claims 1 to 12, wherein the terminal amine-functionalized polysaccharide is carbohydrazide-functionalized hyaluronic acid.

14. The method of any of claims 1 to 13, wherein the crosslinkable polymeric composition comprises o-nitrobenzyl functionalized hyaluronic acid and carbohydrazide-functionalized hyaluronic acid.

15. The method of any of claims 1 to 14, wherein inducing crosslinking of the crosslinkable polymeric composition comprises irradiating the crosslinkable polymeric composition with light from a light source.

16. The method of any of claims 1 to 15, wherein the light source comprises an illumination wavelength of 250 to 500 nm.

17. The method of any of claims 1 to 16, wherein the light source comprises an illumination wavelength of 365 nm.

18. The method of any of claims 2 to 17, wherein the delayed release capsule comprises from 0.1 to 1,000 mg of each TGF-β inhibitor per 100 g of polymer.

19. The method of any of claims 1 to 18, wherein the crosslinkable polymeric composition is provided in a solution.

20. The method of any of claims 1 to 19, wherein the crosslinkable polymeric composition solution is provided at a concentration ranging from 0.1 to 50% by weight of crosslinkable polymeric composition per unit weight of water.

21. The method of any of claims 1 to 20, wherein a ratio of o-nitrobenzyl functionalized hyaluronic acid to carbohydrazide-functionalized hyaluronic acid ranges from 0.01:1 to 100:1.

22. The method of any of claims 1 to 21, wherein a lactic/glycolic acid ratio ranges from 1:1,000 to 1,000:1.

23. The method of any of claims 1 to 22, wherein a lactic/glycolic acid ratio of the o-nitrobenzyl functionalized PLGA is selected to achieve a desired physicochemical characteristic.

24. The method of any of claims 1 to 23, wherein a crosslinkable polymeric composition polymer molecular weight is selected to achieve a desired physicochemical characteristic.

25. The method of any of claims 1 to 24, wherein the crosslinkable polymeric composition polymer end group is selected to achieve a desired physicochemical characteristic.

26. The method of any of claims 1 to 25, wherein the physicochemical characteristic is selected from the group consisting of glass transition temperature, delayed release capsule degradation rate, delayed release capsule release rate, crosslinked polymeric composition degradation rate, and TGF-β inhibitor release rate.

27. The method of any of claims 1 to 26, further comprising covering the dermal tissue with a bandaging material subsequent to administering the scar formation-reducing composition.

28. A composition comprising:
a delayed release capsule comprising at least one TGF-β inhibitor and a crosslinkable polymeric composition.

29. The composition of claim 28, wherein the delayed release capsule comprises an o-nitrobenzyl functionalized polymer.

30. The composition of claim 28 or 29, wherein the o-nitrobenzyl functionalized polymer is o-nitrobenzyl functionalized poly(lactic acid).

31. The composition of any of claims 28 to 30, wherein the o-nitrobenzyl functionalized polymer is o-nitrobenzyl functionalized is poly(glycolic acid).

32. The composition of any of claims 28 to 31, wherein the o-nitrobenzyl functionalized polymer is o-nitrobenzyl functionalized poly(lactic-co-glycolic acid) (PLGA).

33. The composition of any of claims 28 to 32, wherein a lactic/glycolic acid ratio ranges from 1:1,000 to 1,000:1.

34. The composition of any of claims 28 to 33, wherein the delayed release capsule further comprises polyvinyl alcohol (PVA).

35. The composition of any of claims 28 to 34, wherein the delayed release capsule further comprises polyethylene glycol (PEG).

36. The composition of any of claims 28 to 35, wherein the at least one TGF-β inhibitor is SB431542.

37. The composition of any of claims 28 to 36, wherein the crosslinkable polymeric composition comprises an o-nitrobenzyl functionalized polysaccharide, polypeptide, protein, or hydrophilic or water-soluble polymer.

38. The composition of any of claims 28 to 37, wherein the o-nitrobenzyl functionalized polysaccharide is o-nitrobenzyl functionalized hyaluronic acid.

39. The composition of any of claims 28 to 38, wherein the crosslinkable polymeric composition further comprises a terminal amine-functionalized polysaccharide, polypeptide, protein, or hydrophilic or water-soluble polymer.

40. The composition of any of claims 28 to 39, wherein the terminal amine-functionalized polysaccharide is carbohydrazide-functionalized hyaluronic acid.

41. The composition of any of claims 28 to 40, wherein the crosslinkable polymeric composition comprises o-nitrobenzyl functionalized hyaluronic acid and carbohydrazide-functionalized hyaluronic acid.

42. The composition of any of claims 28 to 41, wherein a ratio of o-nitrobenzyl functionalized hyaluronic acid to carbohydrazide-functionalized hyaluronic acid ranges from 0.01:1 to 100:1.

43. The composition of any of claims 28 to 42, wherein the delayed release capsule comprises from 0.1 to 1,000 mg of each TGF-β inhibitor per 100 g of polymer.

44. The composition of any of claims 28 to 43, wherein the composition comprises crosslinkable polymeric composition in a concentration ranging from 0.1 to 50% by weight of crosslinkable polymeric composition per unit weight of water.

45. The composition of any of claims 28 to 45, wherein the delayed release capsule comprises two or more TGF-β inhibitors.
